(19) United States
(12) Patent Application Publication
(10) Pub. No.: US 2014/0190820 A1
(43) Pub. Date: Jul. 10, 2014

(54) REUSABLE APPARATUS WITH SPARINGLY SOLUBLE SOLID FOR CLEANING AND/OR DISINFECTING

(71) Applicants: Ashok V. Joshi, Salt Lake City, UT (US); Sai Bhavaraju, West Jordan, UT (US)

(72) Inventors: Ashok V. Joshi, Salt Lake City, UT (US); Sai Bhavaraju, West Jordan, UT (US)

(21) Appl. No.: 13/734,416

(22) Filed: Jan. 4, 2013

(51) Int. Cl. A61L 2/18; C25B 9/00

(52) U.S. Cl. CPC A61L 2/18; C25B 9/00; A61L 2/186
USPC 204/275.1; 428/34.1

(57) ABSTRACT

An apparatus for cleaning and/or disinfecting a surface or object is disclosed. In one embodiment, such an apparatus includes a container that is refillable with water. A sparingly soluble solid is provided in the container and is positioned to contact the water. The sparingly soluble solid slightly dissolves in the water to form a dilute solution that acts as a cleaning and/or disinfecting solution. The sparingly soluble solid is provided in a quantity sufficient to last several refills of the container.

### RELATED APPLICATIONS

This application claims priority to U.S. Provisional Patent No. 61/583,522 filed on Jan. 5, 2012 and entitled Reusable Spray Bottle With sparingly Soluble Solid For Cleaning and/or Disinfecting, which application is incorporated herein by reference.

### FIELD OF THE INVENTION

This invention relates to an apparatus and methods for cleaning and/or disinfecting surfaces and objects.

### BACKGROUND

The global market for cleaning and disinfecting products and equipment is large and growing, on the order of tens of billions of dollars every year. For example, the global market for industrial and institutional cleaning products is forecast to exceed $36.7 billion by the year 2015. The U.S. represents the largest regional market for industrial and institutional cleaning products, with Europe coming in second. Increased safety and health standards in the food and beverage, food service, and health care sectors, where hygienic environments are required, are driving growth for industrial and institutional cleaning products and equipment.

Currently, a wide range of products and equipment are available to clean and disinfect surfaces and objects in residential, industrial, commercial, hospital, hotel, food processing, and restaurant environments. Unfortunately, some of the best products and equipment for cleaning and disinfecting are confined to the commercial or industrial marketplaces due to their increased expense. That is, the small household user typically cannot afford or justify the expense associated with purchasing and maintaining commercial-quality cleaning products and equipment. Thus, although a substantial need exists for cleaning and disinfecting surfaces and objects in residential settings, typical household users may not have the best and most effective products and equipment at their disposal.

In view of the foregoing, what are needed are products and equipment for cleaning and/or disinfecting surfaces and objects in residential and other similar settings. Ideally, such products and equipment will provide results comparable to products and equipment used in industrial and/or commercial settings but without the associated costs. Further needed are products and equipment that are reusable many times without having to replenish the active agents used for cleaning and/or disinfecting. Yet further needed are water-based cleaners as opposed to solvent-based cleaners. Such water-based cleaners may reduce the environmental, safety, and health concerns associated with solvent-based cleaners.

### SUMMARY

The invention has been developed in response to the present state of the art and, in particular, in response to the problems and needs in the art that have not yet been fully solved by currently available apparatus and methods. Accordingly, the invention has been developed to provide apparatus and methods for cleaning and/or disinfecting surfaces and objects. The features and advantages of the invention will become more fully apparent from the following description and appended claims, or may be learned by practice of the invention as set forth hereinafter.

Consistent with the foregoing, an apparatus for cleaning and/or disinfecting a surface or object is disclosed herein. In one embodiment, such an apparatus includes a container that is refillable with water. A sparingly soluble solid is provided in the container and is positioned to contact the water. The sparingly soluble solid slightly dissolves in the water to form a dilute solution that acts as a cleaning and/or disinfecting solution. The sparingly soluble solid is provided in a quantity sufficient to last several refills of the container.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 shows one embodiment of a container with a sparingly soluble solid used to produce a dilute cleaning/disinfecting solution;

FIG. 2 shows one embodiment of a container with a battery-powered electrolyzer incorporated into the container;

FIG. 3 shows one embodiment of a container with a generator-powered electrolyzer incorporated into the container;

FIGS. 4A and 4B show several examples of electrolyzers and exemplary input and output streams; and

FIG. 5 shows one embodiment of an electrochemical cell that may be used to produce “activated” water containing hydrogen peroxide.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

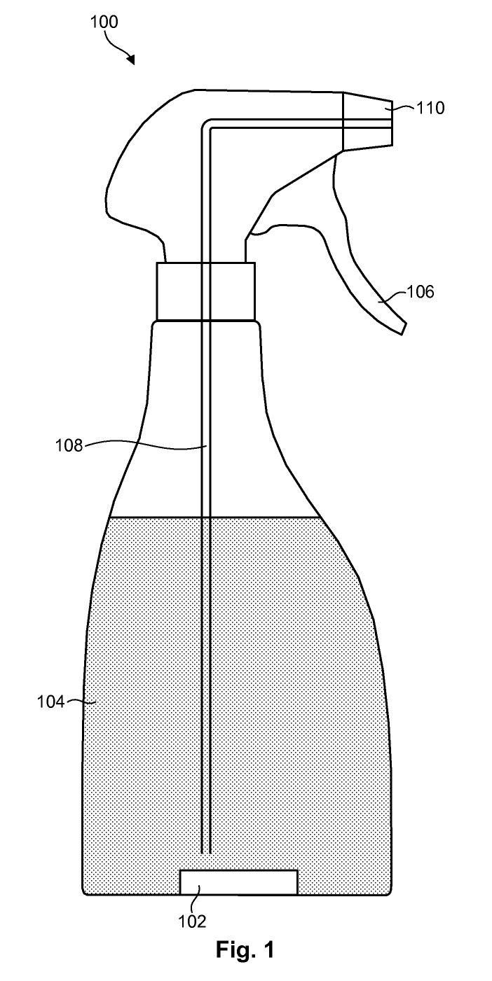

Referring to FIG. 1, one embodiment of a container 100 with a sparingly soluble solid 102 is illustrated. In one embodiment, the container 100 may be a spray bottle 100. As shown, the sparingly soluble solid 102 is placed in or attached to the inside of a container 100 and positioned such as to contact water in the container 100. In certain embodiments, the sparingly soluble solid 102 is provided in the form of a tablet or pellet, which may also contain other ingredients such as surfactants. For the purposes of this description, a “sparingly soluble” solid is defined to be a solid that has a solubility equal to or less than 1 gram per 100 milliliters of water. The sparingly soluble solid 102 is provided in a quantity sufficient to last several refills of the container 100. Each time the container 100 is refilled with water, some fraction of the sparingly soluble solid 102 will dissolve in the water to produce a dilute cleaning and/or disinfecting solution 104.

One benefit of the disclosed container 100 is that, when the solution 104 has been depleted, the user does not have to refill the container 100 with cleaning and/or disinfecting solution, but rather only water. The sparingly soluble solid 102 will be effective to convert the water into a cleaning and/or disinfecting solution 104. Thus, the user will only need to have water at his or her disposal to replenish the container 100 with cleaning and/or disinfecting solution 104.

The properties of the cleaning and/or disinfecting solution 104 will depend on the compounds that are included in the sparingly soluble solid 102. Several exemplary compounds will be discussed in more detail hereafter. The solution 104 may inherently have the cleaning and/or disinfecting properties or exhibit such properties after the solution 104 is passed through an electrolyzer, an electrochemical cell, or both. Embodiments of the container 100 comprising an electrolyzer and/or electrochemical cell will be discussed in more detail hereafter.

The sparingly soluble solid 102 may contain various different compounds to provide the desired disinfecting and/or cleaning properties. For example, in certain embodiments, the sparingly soluble solid 102 contains silver halides, copper halides, bismuth oxyhalides, organic halides, or combinations thereof. In certain other embodiments, moderate to highly soluble chlorides (e.g. NaCl) are incorporated into a sparingly soluble polymer matrix which releases the salts slowly into the water as the polymer slowly dissolves. In other embodiments, the moderate to highly soluble chloride salts are coated with a polymer film that slowly dissolves and releases the salt. In yet other embodiments, the moderate to highly soluble chloride salts are encapsulated. Each of these sparingly soluble compounds, when dissolved in water, produce solutions 106 that have cleaning and/or disinfecting properties. In certain embodiments, the cleaning and/or disinfecting properties of these solutions 106 may be created or enhanced by passing the solutions through an electrolyzer or electrochemical cell. For example, silver chloride (AgCl) is a sparingly soluble solid 102 that may be dissolved in water to generate a dilute solution 104. This solution 104 may be passed through an electrolyzer to dissociate the silver chloride to produce silver ions and chlorine gas and thereby produce chlorinated water. A fraction of the current may also be utilized to split water and thereby generate oxygen and hydrogen. The chlorinated water may be used to clean and/or disinfect a surface or object. The chlorine also produces a scent that, when smelled by a user, reassures the user that cleaning and/or disinfecting is taking place.

As shown in FIG. 1, the container 100 includes a trigger-like actuator 106. Squeezing the trigger 106 actuates a pump (not shown), which draws the solution 104 into a tube 108 and expels the solution through a nozzle 110 onto a surface or object. The cleaning and/or disinfecting agent in the solution 104 may clean by lifting dirt from the surface or object, and/or kill bacteria or other microorganisms residing on the surface or object.

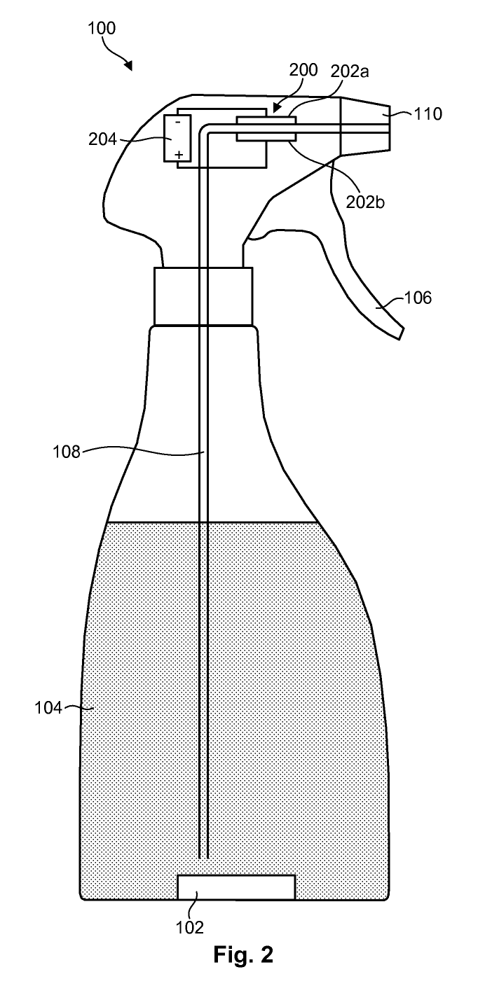

Referring to FIG. 2, as previously mentioned, in selected embodiments, the solution 104 in the container 100 may be passed through an electrolyzer 200 to electrolyze selected compounds in the solution 104. As shown, the electrolyzer 200 includes a pair of electrodes 202a, 202b. A negative electrode 202a attracts positive ions and a positive electrode 202b attracts negative ions. In the illustrated embodiment, a battery 204 creates a potential difference between the electrodes 202a, 202b, resulting in the passage of electrical current between the electrodes 202a, 202b. In certain embodiments, additional circuitry (not shown) may be provided such that the battery 204 only applies a potential difference across the electrodes 202a, 202b when the trigger 106 is actuated and the solution 104 is passed between the electrodes 202a, 202b, thereby preserving energy in the battery 204.

If, for example, the solution 104 is a silver chloride (AgCl) solution, the electrolyzer 200 may disassociate the silver and chlorine in the compound by drawing silver to the negative electrode 202a and chlorine to the positive electrode 202b. The silver will plate the negative electrode 202a, thereby leaving chlorine, a powerful disinfectant, in the exiting stream or spray. A chlorine evolving electrode is used as the positive electrode 202b to generate chlorine. Examples of such chlorine evolving electrodes include Dimensionally Stable Anode (DSA), which is a mixture of ruthenium oxide, iridium oxide, and titanium oxide deposited on titanium metal. Chlorine is effective to kill bacteria or other organisms residing on a surface or object. The chlorine in the exiting stream or spray may also emit a scent that reassures a user that disinfection is taking place. Any residual quantity of silver ions in the delivered stream will also have a disinfecting effect. Silver chloride represents just one example of a compound that may be disassociated by the electrodes 202a, 202b and is not intended to be limiting.

By introducing ions and gases into the solution 104, the electrolyzer 200 is effective to convert the solution 104 into an electrochemically “activated” liquid. For the purposes of this disclosure, an electrochemically “activated” liquid is a liquid with elevated reactivity that contains (1) transient species such as dissolved gases that alter the physical properties of water, and/or (2) reactive species such as ions (hydroxyl, hypochlorite, protons etc.), and/or (3) meta-stable (activated) free radicals formed after exposure to electrochemical energy in the form of a substantial voltage potential or current under non-equilibrium conditions. The term “activated” means, for example, the electrochemical or electrophysical state or condition of having excessive inner potential energy that is attained after exposure to thermodynamically non-equilibrium conditions for a period of time. Meta-stable ions and free radicals relax in time by undergoing a gradual transition from a meta-stable state to a state of thermo-dynamic equilibrium.

In the case of electrochemically activated water, the initial liquid source used to form electrochemically activated water may include, for example, (1) regular, untreated tap water or other water that is commonly available, (2) pure water to which one or more electrolytes have been added, (3) chemically treated tap water, and (4) other aqueous solutions containing a suitable concentration of electrolytes. Examples of suitable electrolytes include chloride salt, nitrate salt, carbonate salt or any other salt that is soluble in water (or other liquid being electrochemically activated). Chloride salts include, for example, sodium chloride (such as pure NaCl), potassium chloride, magnesium chloride, calcium chloride or the like. The term “electrolyte” means any substance that dissociates into two or more ions when dissolved in water or any substance that will conduct an electric current when in solution.

Electrochemically activated water has enhanced cleaning power and sanitizing capability compared to non-electrochemically activated water. Electrochemically activated water also differs from regular or untreated water at the molecular level and electron level. It should also be noted that adding fine gas bubbles to the electrochemically activated water creates a cleaning liquid that can efficiently wet a surface. If a reactive gas is used, such as oxygen, the oxygen gas bubbles may improve the wetting properties of the liquid by reducing the surface tension of the liquid and can be reactive to further enhance the cleaning and/or sanitizing properties of the liquid. The end result is an electrochemically activated foam, froth, or reactive gas with enhanced cleaning and/or sanitizing power.

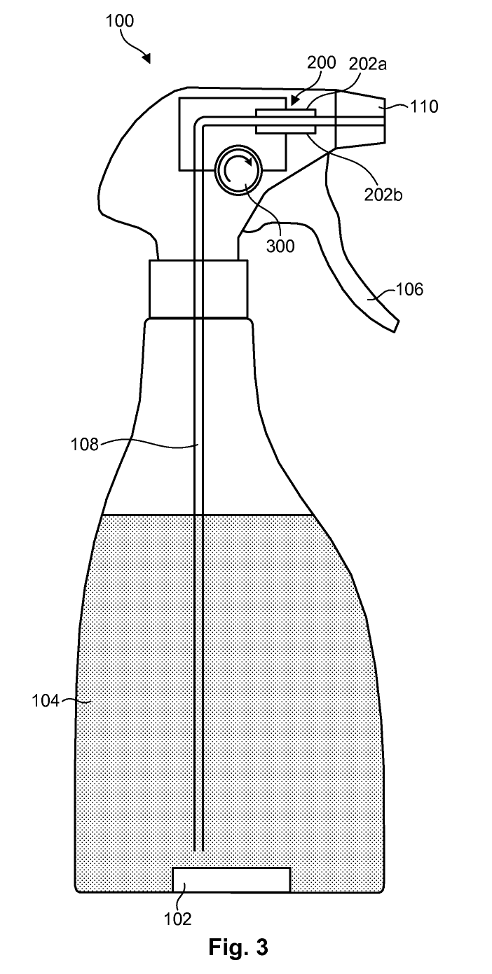

Referring to FIG. 3, in another embodiment, the electrolyzer 200 may be powered by a generator 300, such as a generator 300 actuated by the trigger 106. More particularly, actuating the trigger 106 may cause the generator 300 to spin and create electricity. In certain embodiments, a flywheel may be coupled to the generator 300 such that, when a user spins the generator 300, the flywheel will keep the generator 300 spinning for some designated period of time, such as a second or two. In other embodiments, the generator 300 may spin sufficiently without a flywheel. When a user squeezes the trigger 106, two actions may occur simultaneously: first, power will be supplied to the electrodes 202a, 202b, thereby decomposing selected compounds in the solution 104; second, a pump is driven to emit a stream or spray, containing the disassociated ions, from the nozzle 110.

The container 100 of FIG. 3 is advantageous in that it does not require a battery 204 that may need to be periodically replaced or recharged. Furthermore, the generator 300 only produces power when needed—when the solution is being sprayed from the container 100. The mechanical energy generated by squeezing the trigger 106 provides the power needed to both spray and electrolyze the solution 104 simultaneously.

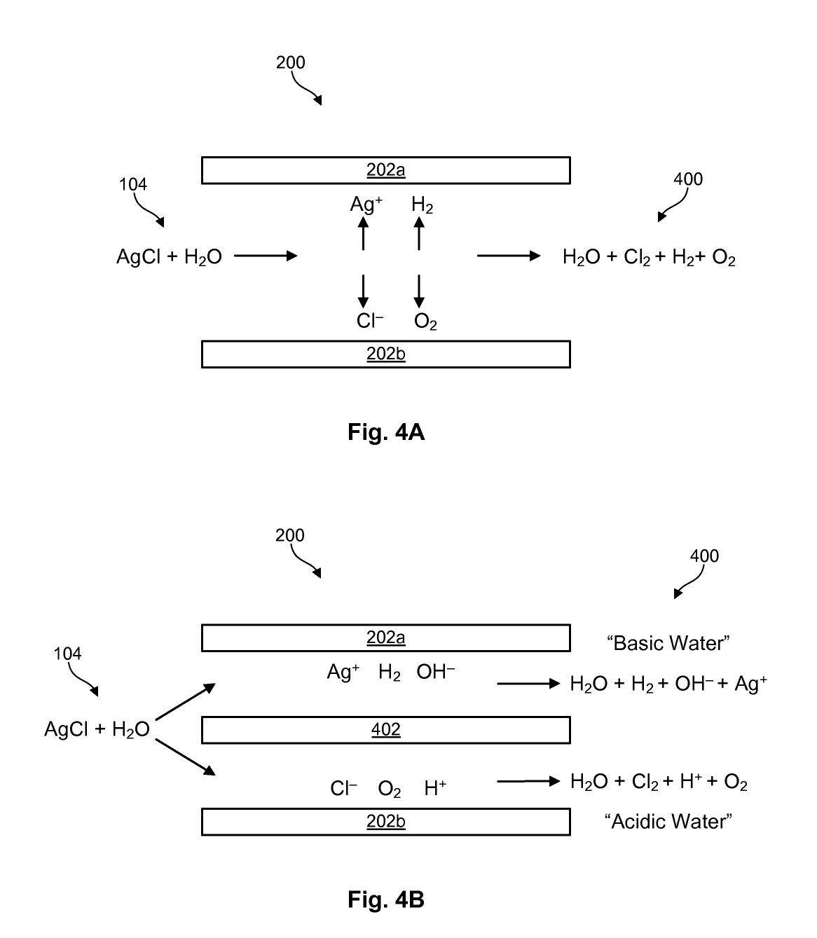

Referring to FIG. 4A, as discussed above, the cleaning and/or disinfecting solution 104 may, in certain embodiments, be passed through an electrolyzer 200 to create or enhance the cleaning and/or disinfecting properties of the solution 104. FIG. 4A shows one example of an electrolyzer 200 receiving a solution 104 of water and silver chloride (AgCl). As shown, upon receiving the solution 104, the electrodes 202a, 202b decompose the silver chloride compound. The negative electrode 202a attracts silver ions (Ag⁺) and the positive electrode 202b attracts chlorine ions (Cl⁻). The silver plates the negative electrode 202a while the positive electrode 202b generates chlorine-based mixed oxidants. The chlorine-based mixed oxidants include predominantly chlorine gas (Cl₂) but also hypochlorite, chlorine dioxide, chlorate, perchlorate and other oxidized chlorine-containing species, which leave the electrolyzer 200 in the exiting stream 400. In certain embodiments, the electrolyzer 200 may also decompose water in the solution 104 to generate some hydrogen, oxygen, and ozone gas in the exiting stream 400. When chlorine gas (Cl₂) combines with water in the exiting stream 400, a dilute mixture of hypochlorous acid (HOCl) and hydrochloric acid (HCl) may be generated in accordance with the following equation:

Cl₂+H₂O→HOCl+HCl

Both hypochlorous acid (HOCl) and hydrochloric acid (HCl) have antimicrobial properties and are commonly used for cleaning and/or disinfecting. Any ozone generated also has disinfecting properties.

Referring to FIG. 4B, in certain embodiments, an ion-exchange membrane 402 may be present between the electrodes 202a, 202b to divide the cell 200 into anode and cathode compartments. If the ion-exchange membrane 402 is of anionic type, then it only allows anions to migrate from the negative electrode 202a to the positive electrode 202b. If the ion-exchange membrane 402 is of cationic type, then it only allows cations to migrate from the positive electrode 202b to the negative electrode 202a. Examples of cationic membranes 402 include NaSICON and Nafion® and examples of anionic membranes 402 include ACS (from Tokuyama corp., Japan) and AMI (from Membranes International). The advantage of this embodiment compared to the embodiment of FIG. 4A is that acidic water is generated in the anode compartment and basic water is generated in the cathode compartment. In the case where no membrane 402 is present, the acidic and basic waters combine in the electrolyzer 200. The acidic and basic streams can be separately delivered to the area to be cleaned where they can combine. Thus water with more ions can be generated when a membrane 402 is present resulting in stronger cleaning and disinfecting action.

It should be recognized that the chemical reactions presented in FIGS. 4A and 4B are presented only by way of example and not limitation. The input stream 106 may contain different compounds which may, in turn, produce different compounds or elements in the output stream 400. FIGS. 4A and 4B simply show how an electrolyzer 200 may be used to alter the chemical properties of a solution 104 to enhance or change the disinfecting/cleaning properties of the solution 104.

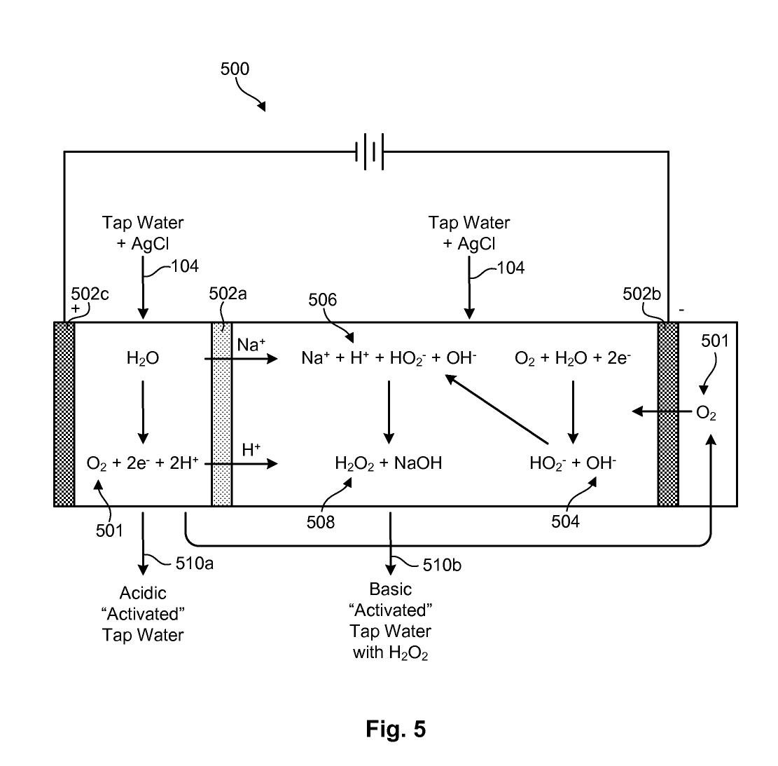

Referring to FIG. 5, one embodiment of an electrochemical cell 500 for producing “activated” water containing hydrogen peroxide is illustrated. Such an electrochemical cell 500 may be used in place of or in conjunction with the electrolyzer 200 previously described. The electrochemical cell 500 may also be considered an electrolyzer 200 for the purposes of the specification and claims.

FIG. 5 shows an electrochemical cell 500 that produces “activated water” which contains hydrogen peroxide, a well-known disinfectant. As shown, in one embodiment, the electrochemical cell 500 receives a solution 104 of tap water and silver chloride. The tap water may contain minerals and salts such as sodium chloride. Upon receiving the tap water, a water splitting reaction occurs at the anode 502c resulting in the generation of oxygen gas 501 and protons. The protons and sodium ions are transported through an ionically conductive membrane 502a. The oxygen gas 501 generated at the anode 502c is then transported to a Gas Diffusion Electrode (GDE) 502b (acting as a cathode 502b) where it reduces to form peroxide ions 504. The construction of a GDE is well known to those skilled in the art. The peroxide ions 504 then react with hydrogen ions 506 (which have been previously transported through the membrane 502a) to produce hydrogen peroxide 508 (H₂O₂). The electrochemical cell 500 produces two output streams 510a, 510b: (1) acidic “activated” tap water with chlorine-based mixed oxidants; and (2) basic “activated” tap water with hydrogen peroxide. These two output streams 510a, 510b may be independently or in combination sprayed from the container 100, along with any chlorinated water produced from the silver chloride (AgCl), to clean and/or disinfect a surface or object.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. An apparatus for cleaning and/or disinfecting a surface or object, the apparatus comprising:
a container that is refillable with water; and
a sparingly soluble solid in the container and positioned to contact the water, wherein the sparingly soluble solid slightly dissolves in the water to form a dilute solution, and the sparingly soluble solid is provided in a quantity sufficient to last several refills of the container.

2. The apparatus of claim 1, wherein the sparingly soluble solid comprises a compound selected from the group consisting of a silver halide, a copper halide, a bismuth oxyhalide, and an organic halide.

3. The apparatus of claim 2, wherein the sparingly soluble solid comprises silver chloride (AgCl).

4. The apparatus of claim 2, wherein the sparingly soluble solid comprises bismuth oxychloride.

5. The apparatus of claim 1, wherein the sparingly soluble solid is provided in the form of a pellet.

6. The apparatus of claim 1, wherein the container is further configured to pass the solution through an electrolyzer to at least partially electrolyze the solution to produce sterilizing material.

7. The apparatus of claim 6, wherein the sterilizing material is one of a halogen and silver ions.

8. The apparatus of claim 6, wherein the sterilizing material is ozone.

9. The apparatus of claim 6, wherein the sterilizing material comprises halogen-based mixed oxidants comprising at least one of the following: hypohalite, halogen dioxide, halate, and perhalate ions.

10. The apparatus of claim 6, wherein the electrolyzer is powered by one of a primary and a rechargeable battery.

11. The apparatus of claim 6, wherein the electrolyzer is powered by a generator that is mechanically driven by a user.

12. The apparatus of claim 11, further comprising an actuator to simultaneously drive the generator and spray the dilute solution from the container.

13. The apparatus of claim 6, wherein the electrolyzer is further configured to at least partially decompose water passing through the electrolyzer.

14. The apparatus of claim 6, wherein the electrolyzer is further configured to convert the water into “activated” water.

15. The apparatus of claim 14, wherein the “activated” water contains hydrogen peroxide.

16. The apparatus of claim 1, wherein the container outputs halogenated water.

17. The apparatus of claim 16, wherein the container outputs chlorinated water.

18. The apparatus of claim 17, wherein the chlorinated water comprises at least one of the following chlorine-based mixed oxidants: hypochlorite, chlorine dioxide, chlorate, and perchlorate ions.

19. The apparatus of claim 1, further comprising a surfactant in the container to contact the water.

20. The apparatus of claim 19, wherein the sparingly soluble solid and surfactant are combined in a pellet.
